(19) United States
(12) Patent Application Publication
(10) Pub. No.: US 2022/0050109 A1
(43) Pub. Date: Feb. 17, 2022

(54) METHOD OF DETECTING CANCER AND/OR TUBERCULOSIS

(71) Applicant: NATIONAL UNIVERSITY OF SINGAPORE, SINGAPORE (SG)

(72) Inventors: Zhunan JIA, Singapore (SG); Venky Thirumalai VENKATESAN, Singapore (SG); Pyng LEE, Singapore (SG)

(21) Appl. No.: 17/297,814

(22) PCT Filed: Nov. 28, 2019
(86) PCT No.: PCT/SG2019/050586

§ 371 Date: May 27, 2021

(30) Foreign Application Priority Data
Nov. 18, 2018 (SG) 10201810657R

(51) Int. Cl. G01N 33/574; G01N 33/569; G01N 33/497

(52) U.S. Cl. CPC G01N 33/57423; G01N 33/5695; G01N 2560/00; G01N 2333/35; G01N 2033/4975; G01N 33/497

(57) ABSTRACT

The invention relates to a method of detecting the presence of cancer in a subject based on a panel of biomarkers comprising Hexanal, Heptanal, Octanal, Decanal, Benzaldehyde, Phenylacetaldehyde, Undecane and Benzoic acid in exhaled breath obtained from a subject. The disclosure also teaches a method of detecting tuberculosis and to distinguish the detection of tuberculosis from cancer or to distinguish the detection of cancer from tuberculosis.

### FIELD

The present disclosure relates to the field of detection of cancer and/or tuberculosis. In particular, the disclosure teaches a method of detecting the presence of cancer in a subject based on a panel of biomarkers in exhaled breath. The disclosure also teaches a method of detecting tuberculosis and distinguishes detection of tuberculosis from the detection of cancer.

### BACKGROUND

Lung cancer is the leading cause of cancer-related deaths globally, claiming 1.6 million deaths each year. The majority of lung cancer cases are diagnosed at a late stage when treatment can no longer provide a cure. More than two thirds of symptomatic patients have lymph nodes or distant metastasis at presentation. Early detection of lung cancer is difficult since clinical symptoms are often not seen until the disease has reached an advanced stage. Early detection of lung cancer is currently aided by computed tomography of the chest, analysis of the type of cells in sputum and bronchoscopic examination of the bronchial passages.

Treatment regimens are determined by the type and stage of the cancer, and include surgery, radiation therapy and/or chemotherapy. In spite of considerable research into therapies for the disease, lung cancer remains difficult to treat. The 5 year survival rate of stage 3 lung cancer is only 23% and that for stage 1 lung cancer is 70%. Therefore, it is critical for lung cancer management to be able to detect the cancer early.

In Singapore, lung cancer is the top killer in men and second in women. There are 46/100,000 people new cases of tuberculosis (TB) yearly in Singapore, and TB may be misdiagnosed as lung cancer. It is therefore important to develop novel techniques, which can discriminate TB from lung cancer. Accordingly, there is a need to overcome, or at least to alleviate, one or more of the above mentioned problems.

### SUMMARY OF THE INVENTION

Disclosed herein is a method of detecting the presence or likelihood of a cancer in a subject, the method comprising the step of detecting or measuring a panel of biomarkers comprising Hexanal, Heptanal, Octanal, Decanal, Benzaldehyde, Phenylacetaldehyde, Undecane and Benzoic acid in a sample obtained from the subject.

Disclosed herein is a method of detecting and treating a cancer in a subject, the method comprising the steps of a) detecting and measuring a panel of biomarkers comprising Hexanal, Heptanal, Octanal, Decanal, Benzaldehyde, Phenylacetaldehyde, Undecane and Benzoic acid in a sample obtained from the subject, and b) administering an anti-cancer therapy to the subject found to have cancer or likely to have cancer.

Disclosed herein is a method of detecting the presence or likelihood of tuberculosis in a subject, the method comprising the step of detecting or measuring a panel of biomarkers comprising Hexanal, Heptanal, Octanal, Decanal, Benzaldehyde, Phenylacetaldehyde, Undecane and Benzoic acid in a sample obtained from the subject.

Disclosed herein is a method of detecting and treating tuberculosis in a subject, the method comprising the steps of: a) detecting or measuring a panel of biomarkers comprising Hexanal, Heptanal, Octanal, Decanal, Benzaldehyde, Phenylacetaldehyde, Undecane and Benzoic acid in a sample obtained from the subject; and b) administering an anti-tuberculosis therapy to the subject found to have tuberculosis or likely to have tuberculosis.

Disclosed is a method of differentiating the presence of tuberculosis from cancer in a subject, the method comprising the step of detecting or measuring a panel of biomarkers comprising Hexanal, Heptanal, Octanal, Decanal, Benzaldehyde, Phenylacetaldehyde, Undecane and Benzoic acid in a sample obtained from the subject.

Disclosed herein is a method of differentiating the presence of cancer from tuberculosis in a subject, the method comprising the step of detecting or measuring a panel of biomarkers comprising Hexanal, Heptanal, Octanal, Decanal, Benzaldehyde, Phenylacetaldehyde, Undecane and Benzoic acid in a sample obtained from the subject.

### BRIEF DESCRIPTION OF THE DRAWINGS

Some embodiments of the present invention will now be described by way of non-limiting example only, with reference to the accompanying drawings in which:

FIG. 1: TIC of representative samples. (1: Acetone; 2: Acetic acid; 3: Dimethylsilanediol; 4: 4-Ethylbenzamide; 5: Hexanal; 6: Oxime-, methoxy-phenyl; 7: Heptanal; 8: Benzaldehyde; 9: Phenol; 10: Octanal; 11: Ethylhexanol; 12: D-Limonene; 13: Benzeneacetaldehyde; 14: Acetophenone; 15: Undecane; 16: Benzoic acid; 17: Decanal; 18: Undecanal; 19: Dodecanal; 20: 1-Dodecanol; 21: 1,2-15,16-Diepoxyhexadecane; 22: 2,4-Di-tert-butylphenol; 23: cis-9-Hexadecenal; 24: p-tert-Octylphenol Hexadecenal; 25: Compounds from GC/MS column).

FIG. 2: a) OPLS-DA score plots of discovery set. b). Color coded map of 13 statistically differential compounds in discovery set.

FIG. 3. ROC curve of combined 8 biomarkers in a) discovery set and b) validation set.

FIG. 4. Data Analysis work flow for biomarker screening.

FIG. 5. OPLS-DA score plot of validation set.

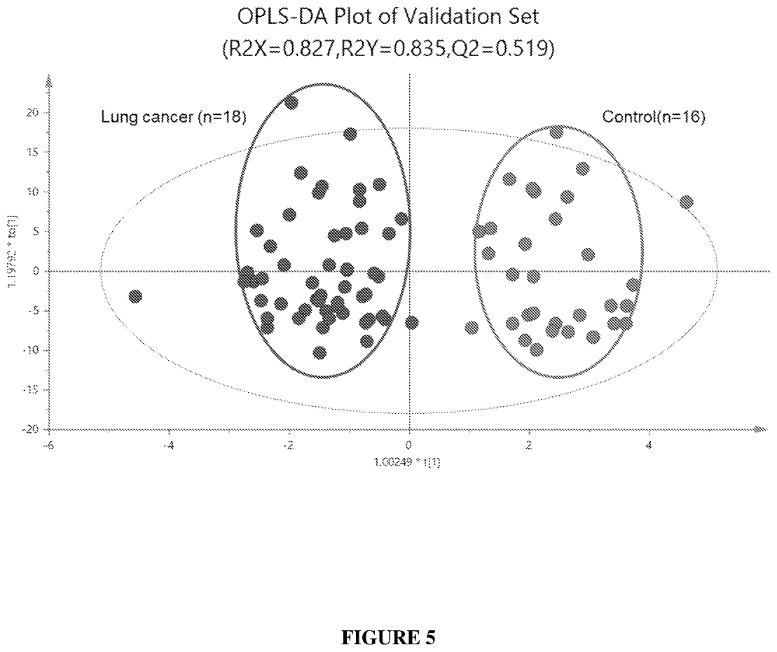

FIG. 6. Representative GC-MS Chromatogram showing good analytical reproducibility between triplicate analyses of a lung cancer patient's breath.

### DETAILED DESCRIPTION

The present disclosure teaches a method of detecting the presence or likelihood of a cancer in a subject. The method may comprise detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid. The panel of biomarkers may be one as shown in Table 6. In one embodiment, there is provided a method of detecting the presence or likelihood of a cancer in a subject, the method comprising detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid. The method may comprise detecting or measuring a panel of biomarkers comprising Hexanal, Heptanal, Octanal, Decanal, Benzaldehye, Phenylacetaldehyde, Undecane and Benzoic acid in a sample obtained from the subject. The method may comprise detecting or measuring a panel of biomarkers as shown in Table 7. The method may comprise detecting or measuring a panel of biomarkers comprising Hexanal, Heptanal, Octanal, Decanal, Benzaldehyde, Phenylacetaldehyde, Undecane, Benzoic acid, acetophenone, dodecanal, acetic acid, phenol and acetone in a sample obtained from the subject. An increase or decrease of the level of each of the biomarker as compared to a control may indicates the presence or likelihood of a cancer in the subject.

Disclosed herein is a method of detecting the presence or likelihood of a cancer in a subject, the method comprising the step of detecting or measuring a panel of biomarkers comprising Hexanal, Heptanal, Octanal, Decanal, Benzaldehye, Phenylacetaldehyde, Undecane and Benzoic acid in a sample obtained from the subject. In one embodiment, there is provided a method of detecting the presence of a cancer in a subject, the method comprising the step of detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increased level of each of the biomarker as compared to a control indicates the presence of a cancer in the subject.

In one embodiment, the method further comprising detecting a biomarker selected from the group consisting of i) Acetophenone, ii) Dodecanal, iii) Acetic acid, iv) Phenol and v) Acetone.

In one embodiment, the method comprises detecting a panel of biomarkers as shown in Table 7.

In one embodiment, there is provided a method of detecting the presence or likelihood of a cancer in a subject, the method comprising the step of detecting or measuring a panel of biomarkers comprising Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject.

In one embodiment, there is provided a method of detecting the presence or likelihood of a cancer in a subject, the method comprising the step of detecting or measuring a panel of biomarkers comprising Hexanal, Heptanal, Octanal, Decanal, Benzaldehyde, Phenylacetaldehyde, Undecane, Benzoic acid, acetophenone, dodecanal, acetic acid, phenol and acetone in a sample obtained from the subject, wherein an increased level of Hexanal, Heptanal, Octanal, Decanal, Dodecanal, Benzaldehyde, Phenylacetaldehyde, phenol, acetophenone, Undecane, acetic acid and Benzoic acid, and a decreased level of acetone, as compared to a control indicates the presence or likelihood of cancer in the subject.

In one embodiment, an increased level of Hexanal, Heptanal, Octanal, Decanal, Dodecanal, Benzaldehye, Phenylacetaldehyde, Phenol, Acetophenone, Undecane, Acetic acid and Benzoic acid, and a decreased level of acetone, as compared to a control indicates the presence or likelihood of cancer in the subject.

The method as defined herein may comprise determining the likelihood of cancer in the subject. The term “likelihood of cancer” may refer to how likely it is for a cancer to be present in a subject. An increased or decreased level of each biomarker in the panel of biomarkers as compared to a control may indicate a more than 10%, 20%, 30%, 40%, 50%, 60%, 70%, 80%, 90% or 99% likelihood of the presence of cancer in the subject.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a sub-stage of a Stage II cancer. One skilled in the art will appreciate that the classification of a Stage II cancer as either an early stage cancer or a late stage cancer depends on the particular type of cancer.

The cancer may, for example, be a cancer is selected from the group consisting of colon cancer, colorectal cancer, gastric cancer, pancreatic cancer, breast cancer, esophageal cancer and lung cancer.

In one embodiment, the cancer is a lung cancer. The lung cancer may be small cell lung cancer (SCLC) or non-small cell lung cancer (NSCLC).

In one embodiment, the method of detecting the presence of a cancer further comprises detecting a symptom of cancer. One of ordinary skill in the medical or veterinary arts is trained to recognize whether a subject has a symptom of cancer. For example, routine testing and/or clinical or veterinary diagnostic evaluation will reveal whether the subject has any of the symptoms of cancer. Common symptoms of cancer include, for example, abnormal bodily function, abnormal tissue growth, fatigue, fever, obstruction of a bodily passageway, pain, and weight loss.

The method of detecting the presence of a cancer may take into account other factors such as family history and risk profiles such as having a history of smoking or a genetic disposition to develop cancer. The method may further comprise taking a biopsy from the subject to determine presence or upregulation of a cancer marker using techniques such as histology or PCR.

In one embodiment, the method further comprises administering an anti-cancer therapy to the subject. The anti-cancer therapy may include surgery, chemotherapy, radiation therapy, a targeted therapy, immunotherapy, or a combination thereof. The targeted therapy may, for example, include the use of gefitinib, erlotinib, or a combination thereof.

The term “administering” refers to contacting, applying or providing a composition of the present invention to a subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates. The “subject” may include a person, a patient or individual, and may be of any age or gender.

As used herein, the term “sample” includes tissues, cells, body fluids and isolates thereof etc., isolated from a subject, as well as tissues, cells and fluids etc. present within a subject (i.e. the sample is in vivo). Examples of samples include: whole blood, blood fluids (e.g. serum and plasm), lymph and cystic fluids, sputum, stool, tears, mucus, hair, skin, breath (e.g. exhaled breath), ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

The method as defined herein may comprise obtaining exhaled breath from a subject. In one embodiment, the sample is exhaled breath. In one embodiment, the sample is end-tidal breath.

Volatile Organic Compounds (VOCs) from the breath of a subject may be collected in a sample, e.g., on a filter, either directly or indirectly. The breath samples may be collected in a collection device which may include sorbent tubes, tedlar bags, canisters etc. It can also comprise collecting samples through a real-time breath sampler. In some embodiments, the breath sample is directly obtained from a subject at or near the laboratory or location where the biological sample will be analyzed. In other embodiments, the breath sample may be obtained by a third party and then transferred, e.g., to a separate entity or location for analysis.

In other embodiments, the sample may be obtained and tested in the same location using a point-of care test. In these embodiments, said obtaining refers to receiving the sample, e.g., from the patient, from a laboratory, from a doctor's office, from the mail, courier, or post office, etc. In some further aspects, the method may further comprise reporting the determination or test results to the subject, a health care payer, an attending clinician, a pharmacist, a pharmacy benefits manager, or any person that the determination or test results may be of interest.

The detection of the volatile organic compounds or biomarkers as defined herein may be detecting using an analytical instrument. Examples of analytical instruments include GC-MS (gas chromatography mass spectrometry), PTR-MS (proton transfer reaction mass spectrometry), SIFT-MS (selected ion flow tube mass spectrometry), sensor technologies.

The term “panel of biomarkers” may refer to two or more biomarkers.

As used herein, the term “increase” or “increased” with reference to a biomarker refers to a statistically significant and measurable increase in the biomarker as compared to a control or reference. The increase is preferably an increase of at least about 10%, or an increase of at least about 20%, or an increase of at least about 30%, or an increase of at least about 40%, or an increase of at least about 50%.

In one embodiment, an increased level of each of the biomarker as compared to a control indicates the presence of a cancer in the subject. The increase in level may be an increase of 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 fold, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times or 100 times or anywhere in between as compared to a control.

As used herein, the term “decrease” or “decreased” with reference to a biomarker refers to a statistically significant and measurable increase in the biomarker as compared to a control or reference. The decrease is preferably a decrease of at least about 10%, or a decrease of at least about 20%, or a decrease of at least about 30%, or a decrease of at least about 40%, or a decrease of at least about 50%.

In one embodiment, a decreased level of each of the biomarker as compared to a control indicates the presence of a cancer in the subject. The decrease in level may refer to a biomarker having 0.9 times or less, 0.8 times or less, 0.7 times or less, 0.6 times or less, 0.5 times or less, 0.4 times or less, 0.3 times or less, 0.2 times or less, 0.1 times or less or anywhere in between as compared to the level of a control.

The control may be a sample obtained from a subject who is healthy. The control may also be samples obtained from a group of subjects who are healthy. Each subject may be one who does not have cancer and/or tuberculosis, whose breath data is used as a reference.

The method as defined herein may comprise determining a weighted score based on the level of each biomarker in the panel of biomarkers in the sample and comparing it to a weighted score obtained from a control sample. Alternatively, the weighted score on the level of each biomarker in the panel of biomarkers in the sample may be compared to a pre-determined value.

In one embodiment, an increased or decreased level of each of the biomarker as compared to a control indicates the presence of cancer or an increased likelihood of cancer in the subject. A subject may have cancer (such as lung cancer) or an increased likelihood of cancer (such as lung cancer) if 3 or more of the following conditions are met:
a) Hexanal concentration is at least 1.2 times of the control;
b) Heptanal concentration is at least 1.5 times of the control;
c) Octanal concentration is at least 1.6 times of the control;
d) Decanal concentration is at least 1.7 times of the control;
e) Dodecanal concentration is at least 1.3 times of the control;
f) Benzaldehyde concentration is at least 1.2 times of the control;
g) Phenylacetaldehyde concentration is at least 1.2 times of the control;
h) Phenol concentration is at least 1.1 times of the control;
i) Acetophenone concentration is at least 1.2 times of the control;
j) Undecane concentration is at least 1.3 times of the control;
k) Acetic acid concentration is at least 1.2 times of the control;
l) Acetone concentration is below 0.9 times of the control; or
m) Benzoic Acid concentration is at least 2 times of the control.

Data analysis algorithm entailing the above conditions with different weightage can be applied to determine the presence or likelihood of having cancer.

In one embodiment, there is provided a method of detecting and treating a cancer in a subject, the method comprising the steps of a) detecting and measuring a panel of biomarkers selected from the group consisting of Hexanal, Heptanal, Octanal, Decanal, Dodecanal, Benzaldehye, Phenylacetaldehyde, Phenol, Acetophenone, Undecane, Acetic acid, Acetone and Benzoic acid in a sample obtained from the subject, and b) administering an anti-cancer therapy to the subject found to have cancer.

In one embodiment, there is provided a method of detecting and treating a cancer in a subject, the method comprising the steps of a) detecting and measuring a panel of biomarkers comprising Hexanal, Heptanal, Octanal, Decanal, Benzaldehye, Phenylacetaldehyde, Undecane and Benzoic acid in a sample obtained from the subject; and b) administering an anti-cancer therapy to the subject.

In one aspect, there is provided a method of detecting and treating a cancer in a subject, the method comprising the steps of a) detecting and measuring a panel of biomarkers comprising Hexanal, Heptanal, Octanal, Decanal, Benzaldehye, Phenylacetaldehyde, Undecane and Benzoic acid in a sample obtained from the subject, wherein an increased level of each of the biomarker as compared to a control indicates the presence of a cancer in the subject; and b) administering an anti-cancer therapy to the subject found to have cancer.

In one embodiment, there is provided a method of detecting and treating a cancer in a subject, the method comprising the steps of a) detecting and measuring a panel of biomarkers comprising Hexanal, Heptanal, Octanal, Decanal, Dodecanal, Benzaldehye, Phenylacetaldehyde, Phenol, Acetophenone, Undecane, Acetic acid, Acetone and Benzoic acid in a sample obtained from the subject, and b) administering an anti-cancer therapy to the subject found to have cancer.

In one embodiment, there is provided a method of detecting and treating a cancer in a subject, the method comprising the steps of a) detecting and measuring a panel of biomarkers comprising Hexanal, Heptanal, Octanal, Decanal, Dodecanal, Benzaldehye, Phenylacetaldehyde, Phenol, Acetophenone, Undecane, Acetic acid, Acetone and Benzoic acid in a sample obtained from the subject; wherein an increased level of Hexanal, Heptanal, Octanal, Decanal, Dodecanal, Benzaldehye, Phenylacetaldehyde, Phenol, Acetophenone, Undecane, Acetic acid and, Benzoic acid, and a decreased level of acetone as compared to a control indicates the presence or likelihood of cancer in the subject, and b) administering an anti-cancer therapy to the subject found to have cancer or likely to have cancer.

In one embodiment, there is provided a method of determining the likelihood a cancer in a subject and treating the cancer, the method comprising a) the steps of detecting or measuring a panel of biomarkers comprising Hexanal, Heptanal, Octanal, Decanal, Benzaldehye, Phenylacetaldehyde, Undecane, Benzoic acid, acetophenone, dodecanal, acetic acid, phenol and acetone in a sample obtained from the subject; and b) treating the cancer.

Tuberculosis

The disclosure teaches a method of detecting the presence of tuberculosis in a subject, the method may comprise detecting a panel of biomarkers as shown in Table 6. In one embodiment, there is provided a method of detecting the presence or likelihood of tuberculosis in a subject, the method comprising the step of detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject.

Disclosed herein is a method of detecting the presence of tuberculosis in a subject, the method comprising the step of detecting or measuring a panel of biomarkers comprising Hexanal, Heptanal, Octanal, Decanal, Benzaldehyde, Phenylacetaldehyde, Undecane and Benzoic acid in a sample obtained from the subject.

In one embodiment, the method further comprising detecting a biomarker selected from the group consisting of i) Acetophenone, ii) Dodecanal, iii) Acetic acid, iv) Phenol and v) Acetone.

In one embodiment, the method comprises detecting a panel of biomarkers as shown in Table 7.

In one embodiment, there is provided a method of detecting the presence of tuberculosis in a subject, the method comprising the step of detecting or measuring a panel of biomarkers comprising Hexanal, Heptanal, Octanal, Decanal, Benzaldehyde, Phenylacetaldehyde, Undecane and Benzoic acid in a sample obtained from the subject, wherein an increase level of Benzoic acid and an unchanged level of Hexanal, Heptanal, Octanal, Decanal, Benzaldehye, Phenylacetaldehyde and Undecane as compared to a control indicates the presence of tuberculosis in the subject.

In one embodiment, an increased level of Benzoic acid and an unchanged level of Hexanal, Heptanal, Octanal, Decanal, Benzaldehye, Phenylacetaldehyde and Undecane as compared to a control indicates the presence of tuberculosis in the subject. The increase in the level of Benzoic acid may be an increase of 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times or 100 times or anywhere in between as compared to a control.

The method as defined herein may comprise determining the likelihood of tuberculosis in the subject. The term “likelihood of tuberculosis” may refer to how likely it is for a cancer to be present in a subject. An increase in the panel of biomarkers as compared to a control may indicate a more than 10%, 20%, 30%, 40%, 50%, 60%, 70%, 80%, 90% or 99% likelihood of the presence of tuberculosis in the subject.

In one embodiment, there is provided a method of determining the likelihood of tuberculosis in a subject, the method comprising the steps of detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increase level of Benzoic acid and an unchanged level of Hexanal, Heptanal, Octanal, Decanal, Benzaldehye, Phenylacetaldehyde and Undecane as compared to a control indicates the presence of tuberculosis in the subject.

In one embodiment, there is provided a method of detecting the presence or likelihood of tuberculosis in a subject, the method comprising the step of detecting or measuring a panel of biomarkers comprising Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject.

In one embodiment, there is provided a method of detecting the presence or likelihood of tuberculosis in a subject, the method comprising the step of detecting or measuring a panel of biomarkers comprising Hexanal, Heptanal, Dodecanal, Benzaldehyde, Phenylacetaldehyde, Phenol, Acetophenone, Acetic acid, and Benzoic acid in a sample obtained from the subject.

In one embodiment, a subject may have tuberculosis or an increased likelihood of tuberculosis if any of the following 3 or more conditions are met:
a) Hexanal concentration is at least 1.1 times of the control;
b) Heptanal concentration is at least 1.1 times of the control;
c) Dodecanal concentration is below 0.7 times of the control;
d) Benzaldehyde concentration is below 0.8 times of the control;
e) Phenylacetaldehyde concentration is below 0.7 times of the control;
f) Phenol concentration is below 0.7 times of the control;
g) Acetophenone concentration is at below 0.8 times of the control;
h) Acetic acid concentration is at below 0.7 times of the control; or
i) Benzoic Acid concentration is at least 2 times of the control.

In one embodiment, the subject is found to have cancer or likely to have cancer if 3 or more of the following conditions are met:
a) Hexanal concentration is at least 1.2 times of the control;
b) Heptanal concentration is at least 1.5 times of the control;
c) Octanal concentration is at least 1.6 times of the control;
d) Decanal concentration is at least 1.7 times of the control;
e) Dodecanal concentration is at least 1.3 times of the control;
f) Benzaldehyde concentration is at least 1.2 times of the control;
g) Phenylacetaldehyde concentration is at least 1.2 times of the control;
h) Phenol concentration is at least 1.1 times of the control;
i) Acetophenone concentration is at least 1.2 times of the control;
j) Undecane concentration is at least 1.3 times of the control;
k) Acetic acid concentration is at least 1.2 times of the control;
l) Acetone concentration is below 0.9 times of the control; or
m) Benzoic Acid concentration is at least 2 times of the control.

In one embodiment, an increased level of Hexanal, Heptanal, and benzoic acid and a decreased level of Dodecanal, Benzaldehyde, Phenylacetaldehyde, Phenol, Acetophenone and Acetic acid indicates the presence or likelihood of tuberculosis.

In one embodiment, there is provided a method of detecting the presence or likelihood of tuberculosis in a subject, the method comprising the step of detecting or measuring a panel of biomarkers comprising Hexanal, Heptanal, Dodecanal, Benzaldehyde, Phenylacetaldehyde, Phenol, Acetophenone, Acetic acid, and Benzoic acid in a sample obtained from the subject, wherein an increased level of Hexanal, Heptanal, and benzoic acid and a decreased level of Dodecanal, Benzaldehyde, Phenylacetaldehyde, Phenol, Acetophenone and Acetic acid indicates the presence or likelihood of tuberculosis

Disclosed herein is a method of detecting and treating tuberculosis in a subject, the method comprising the steps of: a) detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject; and b) administering an anti-tuberculosis therapy to the subject found to have tuberculosis or likely to have tuberculosis.

In one embodiment, there is provided a method of detecting and treating tuberculosis in a subject, the method comprising the steps of:

a) detecting or measuring a panel of biomarkers comprising Hexanal, Heptanal, Octanal, Decanal, Benzaldehye, Phenylacetaldehyde, Undecane and Benzoic acid in a sample obtained from the subject; and b) administering an anti-tuberculosis therapy to the subject found to have tuberculosis or likely to have tuberculosis.

In one embodiment, there is provided a method of detecting and treating tuberculosis in a subject, the method comprising the steps of:

a) detecting or measuring a panel of biomarkers comprising Hexanal, Heptanal, Dodecanal, Benzaldehyde, Phenylacetaldehyde, Phenol, Acetophenone, Acetic acid, and Benzoic acid in a sample obtained from the subject; and b) administering an anti-tuberculosis therapy to the subject found to have tuberculosis or likely to have tuberculosis.

In one embodiment, there is provided a method of detecting and treating tuberculosis in a subject, the method comprising the steps of:

a) detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increase level of Benzoic acid and an unchanged level of Hexanal, Heptanal, Octanal, Decanal, Benzaldehye, Phenylacetaldehyde and Undecane as compared to a control indicates the presence of tuberculosis in the subject; and b) administering an anti-tuberculosis therapy to the subject.

In one embodiment, there is provided a method of detecting and treating tuberculosis in a subject, the method comprising the steps of:

a) detecting or measuring a panel of biomarkers comprising Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject; and

b) administering an anti-tuberculosis therapy to the subject found to have tuberculosis or likely to have tuberculosis.

In one embodiment, there is provided a method of determining the likelihood of tuberculosis in a subject and treating the tuberculosis, the method comprising the steps of a) detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increase level of Benzoic acid and an unchanged level of Hexanal, Heptanal, Octanal, Decanal, Benzaldehye, Phenylacetaldehyde and Undecane as compared to a control indicates the presence of tuberculosis in the subject; and b) treating the tuberculosis.

In one embodiment, the method of detecting the presence of a tuberculosis further comprises detecting a symptom of cancer. One of ordinary skill in the medical or veterinary arts is trained to recognize whether a subject has a symptom of tuberculosis. For example, routine testing and/or clinical or veterinary diagnostic evaluation will reveal whether the subject has any of the symptoms of tuberculosis. Common symptoms of tuberculosis include, for example, fatigue, fever, night sweats, chest pain, prolonged coughing.

The anti-tuberculosis therapy may include the use of Isoniazid, Rifampin (Rifadin, Rimactane), Ethambutol (Myambutol), Pyrazinamide or combinations thereof. The anti-tuberculosis therapy may also include therapy for drug-resistant tuberculosis which may involve the use of a combination of antibiotics such as fluoroquinolones and injectable medications, such as amikacin, kanamycin or capreomycin.

Disclosed herein is a method of differentiating the presence of tuberculosis from cancer in a subject, the method comprising the step of detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject. The panel of biomarkers may be a panel as shown in Table 6.

Disclosed herein is a method of differentiating the presence of tuberculosis from cancer in a subject and treating the tuberculosis, the method comprising the steps of a) detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject; and b) treating the tuberculosis.

Provided herein is a method of differentiating the presence of tuberculosis from cancer in a subject, the method comprising the step of detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increase level of Benzoic acid and an unchanged level of Hexanal, Heptanal, Octanal, Decanal, Benzaldehye, Phenylacetaldehyde and Undecane as compared to a control indicates the presence of tuberculosis and not cancer in the subject.

Provided herein is a method of differentiating the presence of tuberculosis from cancer in a subject and treating the tuberculosis, the method comprising the steps of a) detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increased level of Benzoic acid and an unchanged level of Hexanal, Heptanal, Octanal, Decanal, Benzaldehye, Phenylacetaldehyde and Undecane as compared to a control indicates the presence of tuberculosis and not cancer in the subject; and b) treating the tuberculosis.

Disclosed herein is a method of differentiating the presence of tuberculosis from cancer in a subject, the method comprising the step of detecting or measuring a panel of biomarkers comprising Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject.

Disclosed herein is a method of differentiating the presence of tuberculosis from cancer in a subject, the method comprising the step of detecting or measuring a panel of biomarkers comprising Octanal, Decanal, Dodecanal, Benzaldehyde, Phenylacetaldehyde, Phenol, Acetophenone and Acetic acid in a sample obtained from the subject

One or more of the following conditions may be used as index to differentiate cancer (such as lung cancer) and tuberculosis.

(a) Octanal of cancer patients is at least 1.6 times of control group, but Octanal of tuberculosis patients is comparable to control;

(b) Decanal of cancer patients is at least 1.6 times of control group, but Decanal of tuberculosis patients is comparable to control;

(c) Dodecanal of cancer patients is at least 1.3 times of control group. But Dodecanal of tuberculosis patients is below 0.7 times of control;

(d) Benzaldehyde concentration of cancer is at least 1.2 times of the control, but Benzaldehyde concentration of TB is below 0.8 times of the control;

(e) Phenylacetaldehyde concentration of cancer is at least 1.2 times of the control, but Phenylacetaldehyde concentration is below 0.7 times of the control,

(g) Phenol concentration of cancer is at least 1.1 times of the control, but Phenol concentration is below 0.7 times of the control;

(h) Acetophenone concentration of cancer is at least 1.2 times of the control; but Acetophenone concentration of tuberculosis is at below 0.8 times of the control; or

(i) Acetic acid concentration of cancer is at least 1.2 times of the control, but Acetic acid concentration of tuberculosis is at below 0.7 times of the control.

In one embodiment, any one of the following conditions indicates the presence or likelihood of tuberculosis and not cancer:

(a) a comparable level of Octanal to control;

(b) a comparable level of Decanal to control;

(c) a Dodecanal level of less than 0.7 times of the control;

(d) a Benzaldehyde level of less than 0.8 times of the control;

(e) a Phenylacetaldehyde level of less than 0.7 times of the control,

(g) a Phenol concentration of less than 0.7 times of the control;

(h) an Acetophenone concentration of less than 0.8 times of the control; or

(i) an Acetic acid level of less than 0.7 times of the control.

A comparable (or unchanged) level of a biomarker (such as Octanal or Decanal) as compared to a control may refer to a level that is less than 1.1 times and more than 0.9 times (<1.1 times and >0.9 times) of the control.

Disclosed herein is a method of differentiating the presence of cancer from tuberculosis in a subject, the method comprising the step of detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject. The panel of biomarkers may be a panel as shown in Table 6.

Disclosed herein is a method of differentiating the presence of cancer from tuberculosis in a subject and treating the cancer, the method comprising the steps of a) detecting or measuring a panel of biomarkers selected from the group consisting of Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject; and b) treating the cancer in the subject.

Provided herein is a method of differentiating the presence of cancer from tuberculosis in a subject, the method comprising the step of detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increased level of each of the biomarker as compared to a control indicates the presence of a cancer and not tuberculosis in the subject.

Provided herein is a method of differentiating the presence of cancer from tuberculosis in a subject and treating the cancer, the method comprising the steps of a) detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increased level of each of the biomarker as compared to a control indicates the presence of a cancer and not tuberculosis in the subject; and b) treating the cancer.

Disclosed herein is a method of differentiating the presence of cancer from tuberculosis in a subject, the method comprising the step of detecting or measuring a panel of biomarkers comprising Hexanal, Heptanal, Octanal, Decanal, Dodecanal, Benzaldehyde, Phenylacetaldehyde, Phenol, Acetophenone, Undecane, Acetic acid, Acetone and Benzoic acid in a sample obtained from the subject.

Disclosed herein is a method of differentiating the presence of cancer from tuberculosis in a subject, the method comprising the step of detecting or measuring a panel of biomarkers comprising Octanal, Decanal, Dodecanal, Benzaldehyde, Phenylacetaldehyde, Phenol, Acetophenone and Acetic acid in a sample obtained from the subject.

In one embodiment, any one of the following conditions indicates the presence or likelihood of cancer and not tuberculosis:

(a) an Octanal level of at least 1.6 times of the control;

(b) a Decanal level of at least 1.6 times of the control;

(c) a Dodecanal level of at least 1.3 times of the control;

(d) a Benzaldehyde level of at least 1.2 times of the control;

(e) a Phenylacetaldehyde level of at least 1.2 times of the control,

(g) a Phenol concentration of at least 1.1 times of the control;

(h) an Acetophenone concentration of at least 1.2 times of the control; or

(i) an Acetic acid level of at least 1.2 times of control.

In one embodiment, there is provided a method of predicting the presence of cancer in a subject, the method comprising the step of detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increased level of each of the biomarker as compared to a control indicates the presence of a cancer in the subject. The increased level of each of the biomarker as compared to a control may indicate an increased risk or likelihood of the presence of a cancer in a subject.

In one embodiment, there is provided a method of predicting the presence of cancer and treating the cancer in a subject, the method comprising a) the step of detecting or measuring a panel of biomarkers comprising Hexanal, Heptanal, Octanal, Decanal, Benzaldehye, Phenylacetaldehyde, Undecane and Benzoic acid in a sample obtained from the subject, wherein an increased level of each of the biomarker as compared to a control indicates the presence of a cancer in the subject; and b) treating the cancer.

Provided herein is a pharmaceutical composition, comprising an anti-cancer therapy or anti-cancer therapeutic agent. Provided herein is also a pharmaceutical composition comprising an anti-tuberculosis therapy or anti-tuberculosis therapeutic agent.

Also provided herein are kits for detecting the presence of a cancer and/or tuberculosis in a subject. The kit may be configured to detect or measure a panel of biomarkers comprising Hexanal, Heptanal, Octanal, Decanal, Benzaldehye, Phenylacetaldehyde, Undecane and Benzoic acid in a sample obtained from the subject, wherein an increased level of each of the biomarker as compared to a control indicates the presence of a cancer in the subject. The kit may be configured to detect or measure a panel of biomarkers comprising Hexanal, Heptanal, Octanal, Decanal, Benzaldehye, Phenylacetaldehyde, Undecane and Benzoic acid in a sample obtained from the subject, wherein an increase level of Benzoic acid and an unchanged level of Hexanal, Heptanal, Octanal, Decanal, Benzaldehye, Phenylacetaldehyde and Undecane as compared to a control indicates the presence of tuberculosis in the subject.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### Materials and Methods

Study Design

The research was approved by the Institutional Review Boards of National University of Singapore; all subjects gave signed informed consent to participate. 14 lung cancer patients, 14 non-lung cancer controls (patients with other diseases such as COPD, asthma, diabetes and hypertension) and 3 TB patients were recruited for biomarker discovery (Table 1). There is no significant difference in terms of gender, age and smoking history (p>0.05) between lung cancer group and non-lung cancer controls. These two groups also have matched comorbidities.

In subsequent validation set, 18 lung cancer patients and 16 non-lung cancer controls with matched gender, age, smoking history and comorbidities were recruited (Table 2).

In a further validation set, 32 lung cancer patients, 30 non-lung cancer controls (patients with other diseases such as COPD, asthma, diabetes and hypertension) and 3 TB patients were recruited for biomarker discovery. There is no significant difference in terms of gender, age and smoking history (p>0.05) between lung cancer group and non-lung cancer controls. These two groups also have matched comorbidities.

Breath Collection Procedures

The breath samples were collected using BIO-VOC sampler (purchased from Markes International, United Kingdom). The subject was asked to blow through a disposable mouth-piece till mid-tidal breath to completely displace existing environmental air in the sampling tube. The sampling tube stores a fixed volume of end-tidal breath. There is a one-way filter to prevent pathogen contamination from the air. The sampling tube is sealed with a PTFE plug. The sample was then transferred to Tenax Tube (purchased from SKC, United Kingdom) using a plunger. The tenax tube is airtight and has sorbent materials that can trap VOCs. The tenax tubes were stored at 4° C. until analysis. Each tenax tube was bar-coded and had a unique ID for identification of subjects. Three tubes of breath samples were collected form each lung cancer subject and two tubes of breath were collected from each control subject. One environmental air sample was also collected to account for potential environmental contamination.

TD-GC/MS Analysis

The analysis of VOCs was performed by Thermal Desorption-Gas Chromatography mass spectrometry (TD-GC/MS), using a Unity Series 2 Thermal Desorber (Markes International Limited) and a 6890 GC system (Agilent Technologies) interfaced with a 5973 MSD (Agilent Technologies).

The sampling tube was pre-purged for 1 min and the collected breath gases were carried out at 270° C. for 10 min in the stage of primary desorption by helium gas. The total flow rate was set at 60 ml/min. The cold trap was maintained at −10° C. After primary desorption, the cold trap was rapidly heated up from −10° C. to 280° C. and hold on 5 min. The desorbed analytes were injected into a HP-5MS capillary column (60 m×250 μm×0.25 μm nominal; Agilent), via a transfer line at 120° C. The initial GC oven temperature was set at 40° C. Once the sample was injected, the oven temperature was increased to 140° C. at a rate of 5° C./min, increased to 190° C. at a rate of 20° C./min and then increased to 230° C. at a rate of 5° C./min and held for 1 min. Finally, the temperature was increased to 300° C. at a rate of 30° C./min and held for 5 mins. Helium carrier gas flow rate was 2 ml/min. Detection was achieved using MS in electron impact mode and full scan monitoring (33-550 amu). The temperature of the ion source was set at 230° C., and the quadrupole was set at 150° C. The transfer line temperature was 280° C. The gain factor was fixed at 1.58.

Proton-Transfer-Reaction Mass Spectrometry (PTR-MS) Analysis

The above online sampler and procedure is specially designed for proton-transfer-reaction mass spectrometry (PTR-MS) analysis. The online sampler is connected to the PTR-MS to transfer the breath sample to it in real time for analysis. This method effectively avoided sample absorption, storage and transportation, thereby minimizing sample loss and contamination. The method also enables fast on-spot detection which allows point-of-care diagnosis. The PTR-MS measures the concentration of a few hundred VOCs, some of which are disease biomarkers and will be singled out for data analysis. The peaks were identified using the library of PTR-MS, and further validated by external standards.

Statistical Analysis

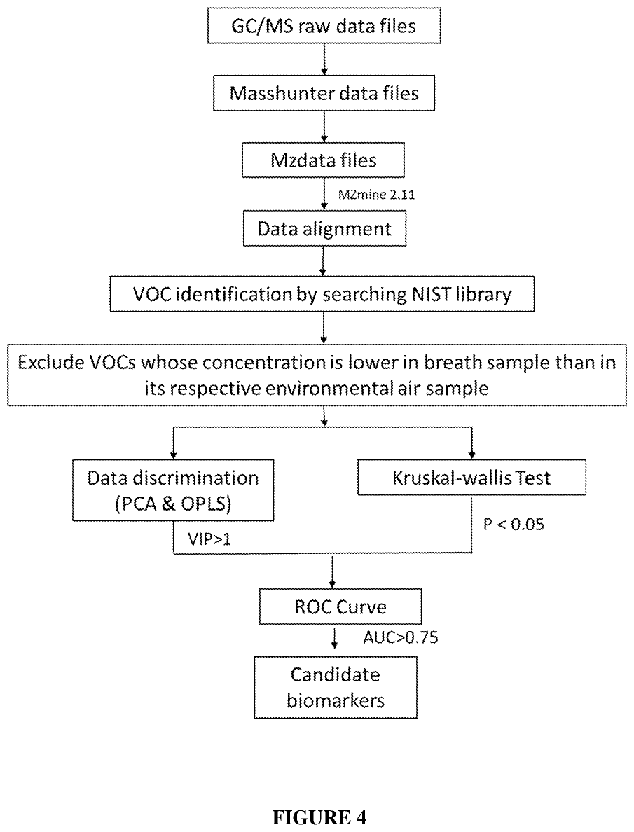

All data files were transferred into Masshunter data by GC MSD Translator software (Agilent Technologies) and then exported into mzdata files, which could be imported into MZmine 2.11 for peak alignment and normalization. VOC identification was conducted by searching NIST library. Only compounds with a match and reverse match score of more than 700 were reported. The VOC concentrations in the samples were also compared with that in the environmental samples. Compounds whose concentration is lower than environmental background were excluded. OPLS-DA (orthogonal projections to latent structures-discriminant analysis), a statistical clustering analysis, was performed on SIMCA-P 13.0 (Umetrics). Kruskal-wallis test and receiver operating characteristic (ROC) analysis were performed on SPSS software (IBM). The data analysis workflow for biomarker screening is shown in FIG. 4.

Three breath samples from each case and two samples from each control subject were taken. During data analysis, we found the variance in different samples from one single subject varies from person to person. Possible reasons are discussed in detail in discussion section. In OPLS-DA analysis, instead of taking average of the multiple samples from one subject, we took each breath as one sample.

### Example 1

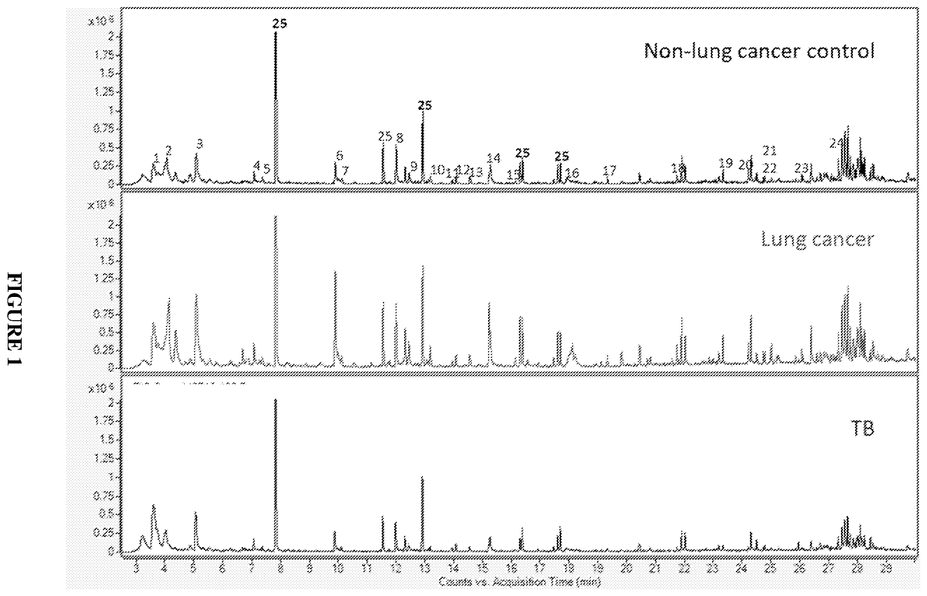

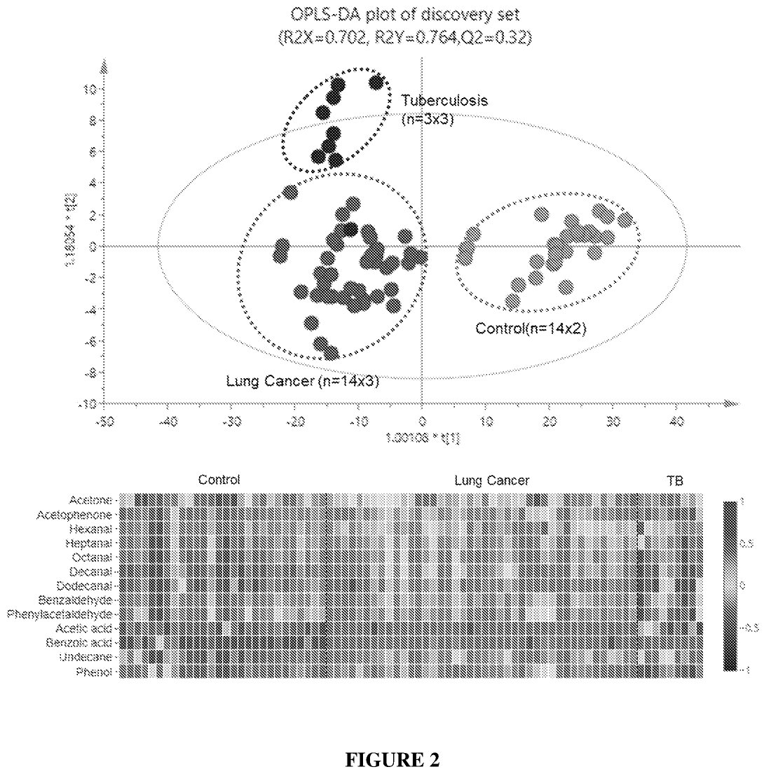

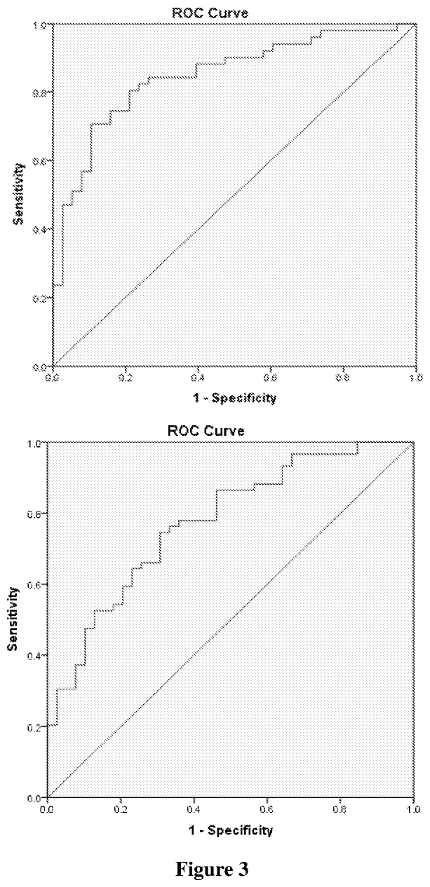

FIG. 1 shows Total Ion Chromatograms (TIC) of a representative sample from each group, each peak is labeled with the compound name. Using kruskal-wallis test and VIP calculation in OPLS-DA model, a total of 13 compounds were found to be statistically different. FIG. 2 a) is the OPLS-DA score plot of the VOCs from discovery set. In this plot, each breath sample is represented by one dot. It shows clear separation between lung cancer and control subjects, as well as between lung cancer and TB patients. The relative concentration of each VOC was linearly normalized into the range of −1 to 1 and plotted in the color coded map shown in FIG. 2.b). A receiver operating characteristics (ROC) analysis was performed using the 13 compounds on discovery set samples. The area under curve (AUC) of each compound was listed in table 4. Out of these 13, 8 compounds with AUC value above 0.75 (Hexanal, Heptanal, Octanal, Benzaldehyde, Undecane, Phenylacetaldehyde, Decanal and Benzoic acid) were selected as combined biomarker and a ROC curve was plotted using them, shown in FIG. 3 a). The combined 8 biomarker achieved an AUC value of 0.85, sensitivity of 88% and specificity of 76%. The validation set data was analyzed in the same way. OPLS-DA score plot of validation set is shown in FIG. 4. Table 3 lists the fold change (lung cancer/control) and p value of the 8 VOCs in both discovery and validation set. In the validation set, the combined 8 biomarker achieved an AUC of 0.78, sensitivity of 80% and specificity of 68% (FIG. 3b).

### Example 2

In this study, seven aldehydes (including hexanal, heptanal, ocatanal, decanal, dodecanal, benzaldehyde, phenyl acetaldehyde) were found in elevated levels in lung cancer patients' breath, as compared to that in non-lung cancer controls. This is possibly due to increased level of Alcohol dehydrogenase (ADH) in cancer tissues. It is believed that ADH is over activated in cancer cells, which means cancer cell has higher ability to oxidize alcohol into aldehydes. Alcohols are products of alkanes by cytochrome p450, alkanes in turn are products of lipid peroxidation, a process which is also up regulated in cancer cells. Hexanal heptanal and octanal were higher in lung cancer patients' breath samples. It is highly likely that these aldehydes are products of cancerous pathological process. Benzenaldehyde was found to be increased in lung cancer cell cultures. An alkane, undecane, was also found to be elevated in this study. Undecane is by product of lipid peroxidation by reactive oxygen species in cancer tissue. Breath acetic acid vapor was used as biomarker for gastro-esophageal reflux disease and cystic fibrosis, but it has not been reported to be associated with lung cancer before. Benzoic acid was found to be higher in both lung cancer patients' breath and TB patients' breath, the origin of benzoic acid in unknown. Increased level of benzoic acid, but not other VOCs, could be used to differentiate TB and lung cancer.

Despite the fact that many studies have produced lists of volatile biomarkers for lung cancer, none of these has been translated into clinics so far. The reason is the inconsistencies in the sampling method and study design.

In the current study, several physiological and pathological confounders including age, gender, smoking history and comorbidities were controlled. Isoprene, alkanes and methylated alkanes were found to be related to age. In many earlier studies, disease groups is significantly older than the control group, this might have biased the results. Gender also has an effect on breath VOC profile. Smoking related VOCs such as acetonitrile and benzene will also affect the results. As one of the biggest risk factors for lung cancer, smoking history between case and control group should be closely matched to avoid any bias. Diseases other than the targeted one will also change the breath VOC profile; therefore it is critical that control and case have matched comorbidities in order to find the VOCs specifically associated with the targeted disease.

During breath sampling, alveolar phase was collected by using a BIO-VOC sampler. For diagnostic purpose, end-tidal breath is desired because they are more representative of the endogenous metabolism, compared to airway dead space air which is often affected by diet and bacteria. Some VOCs (such as carbonic acid, dimethyl ester, methyl formate) were found to be significantly higher in end-tidal breath while methylene chloride and pentane, 3-ethyl were lower in end-tidal breath compared to whole breath. The use of sorbent traps minimizes sample loss and contamination during transportation and storage. Another important confounding factor is the environmental background. Room air especially in clinics may contain high levels of VOCs and they vary from day to day. The degree of influence depends on the type of VOC, time and concentration of exposure etc. Many studies have adopted inspiratory filters to reduce the effect of background VOCs. However, it is not known yet how long does it takes for the human body to equilibrate with filtered clean air. In the current study we did not use an inspiratory filter due to limited resources. Instead, this confounder was controlled by taking a room air sample for each subject at the same time and location. VOC whose concentration is lower in the breath than in the room air were excluded from further analysis.

Study Design

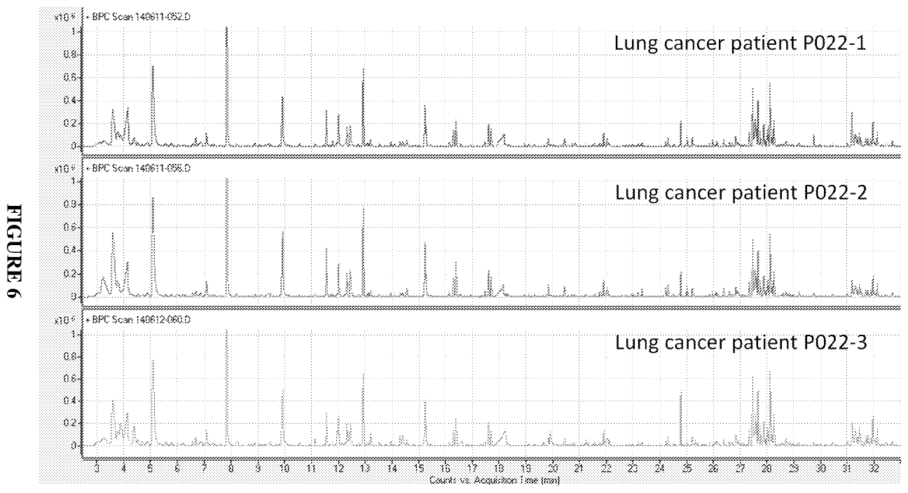

It should be noted that there was no control over diet and exercise of the participants; these could have potential effects on the results. Secondly, three breath samples from each patient and two breath samples from each control subject were collected; the reproducibility of different breath samples from a single person may vary for different subjects. FIG. 6 shows the independent randomized analysis of three breaths collected from one lung cancer patients; it demonstrates good analytical reproducibility, indicating that the analytical methods are consistent and reliable with no significant technical or systemic variations. However, for some other subjects, the reproducibility among different breath samples is poor. This could be due to inconsistent breathing maneuvers. As we had no control over the flow rate, some subjects may have exhaled at more consistent flow rates than other. Studies have shown that expiratory flow rate and breath holding affect the levels of various VOCs including acetone, ethanol, methanol, isoprene, ethane and pentane.

### Example 3

### CONCLUSION

It was concluded that a combined biomarker of 8 VOCs (hexanal, heptanal, octanal, benzaldehyde, undecane, phenyl acetaldehyde, decanal, and benzoic acid) provided a rational detection of lung cancer in both discovery set and validation set. The results also indicate that breath VOC can aide differential diagnosis of TB vs. Lung cancer.

TABLE 1
Clinical characteristics of discovery set.

| | Lung Cancer | Controls | TB |
|---|---|---|---|
| Total Number | 14 | 14 | 3 |
| Sex (M/F) | 10/4 | 10/4 | 2/1 |
| Mean Age ± SD | 67 ± 10 | 67 ± 7 | 51 ± 20 |
| Smoking History | | | |
| Current smokers | 4 | 4 | 2 |
| Ex-smokers | 8 | 8 | 0 |
| Pack-years of smoking | 38.7 | 38.6 | 40 |
| Non-smokers | 2 | 2 | 1 |
| Histology | | | |
| Adenocarcinoma | 9 | | |
| Squamous cell carcinoma | 3 | | |
| Unknown | 2 | | |
| Stage | | | |
| 1 and 2 | 3 | | |
| 3 and 4 | 10 | | |
| Unknown | 1 | | |

TABLE 2
Clinical characteristics of validation set.

| | Lung Cancer | Controls |
|---|---|---|
| Total Number | 18 | 16 |
| Sex (M/F) | 14/4 | 13/3 |
| Mean Age ± SD | 66 ± 9 | 64 ± 12 |
| Smoking History | | |
| Current smokers | 3 | 3 |
| Ex-smokers | 10 | 9 |
| Pack-years of smoking | 37.5 | 30 |
| Non-smokers | 5 | 4 |
| Histology | | |
| Adenocarcinoma | 16 | |
| Squamous cell carcinoma | 1 | |
| Unknown | 1 | |
| Stage | | |
| 1 and 2 | 2 | |
| 3 and 4 | 15 | |
| Unknown | 1 | |

TABLE 3
Fold change (lung cancer/control) and p value
of 8 compounds in discovery set and validation set

| | Discovery Set | | Validation Set | |
|---|---|---|---|---|
| | fold change | p. value | fold change | p value |
| Hexanal | 1.8 | 0.00000001 | 1.3 | 0.05 |
| Heptanal | 2.0 | 0.000005 | 1.2 | 0.27 |
| Octanal | 2.1 | 0.000006 | 1.3 | 0.26 |
| Decanal | 2.4 | 0.000003 | 1.8 | 0.17 |
| Benzaldehyde | 1.7 | 0.000009 | 1.3 | 0.38 |
| Phenylacetaldehyde | 1.4 | 0.001 | 1.3 | 0.03 |
| Undecane | 2.0 | 0.00002 | 1.4 | 0.08 |
| Benzoic Acid | 3.5 | 0.00003 | 1.4 | 0.21 |

TABLE 4
Area under curve for 13 VOCs.

| Test Result Variable(s) | Area |
|---|---|
| Hexanal | 0.825593 |
| Heptanal | 0.796182 |
| Octanal | 0.785088 |
| Benzaldehyde | 0.784056 |
| Undecane | 0.776058 |
| Phenylacetaldehyde | 0.77193 |
| Decanal | 0.766512 |
| Benzoic acid | 0.76161 |
| Acetophenone | 0.747162 |
| Dodecanal | 0.74355 |
| Acetic acid | 0.716202 |
| Phenol | 0.714138 |
| Acetone | 0.538184 |

TABLE 5
List of the 13 VOCs and Ratios Discovered in Clinical Trial

| | LC/Control | LC/TB | TB/Control |
|---|---|---|---|
| Hexanal | 1.801373932 | 1.434408 | 1.255830824 |
| Heptanal | 2.027594142 | 1.687495 | 1.201540595 |
| Octanal | 2.124299933 | 2.107283 | 1.008075251 |
| Decanal | 2.373976514 | 2.535356 | 0.936348208 |
| Dodecanal | 1.946240575 | 3.742809 | 0.519994649 |
| Benzaldehyde | 1.650186284 | 2.308336 | 0.714881408 |
| Phenylacetaldehyde | 1.412278617 | 2.551857 | 0.55343174 |
| Phenol | 1.264441619 | 3.269252 | 0.386767828 |
| Acetophenone | 1.413477326 | 2.290931 | 0.616988099 |
| Undecane | 1.980235072 | 1.903553 | 1.040283729 |
| Acetic acid | 1.840005786 | 3.061764 | 0.600962586 |
| Acetone | 0.855646756 | 0.759819 | 1.126118967 |
| Benzoic Acid | 3.50988421 | 0.884038 | 3.970288715 |

TABLE 6
Combination of Biomarkers

| Statement | Biomarkers |
|---|---|
| 1 | A panel of biomarkers selected from the group consisting of |
| | Hexanal, Heptanal, Octanal, Decanal, Benzaldehyde, |
| | Phenylacetaldehyde, Undecane, Benzoic acid, |
| | acetophenone, dodecanal, acetic acid, phenol and acetone. |
| 2 | The panel of statement 1, wherein the panel comprises 1 |
| | biomarker. |
| 3 | The panel of statement 1, wherein the panel comprises 2 |
| | biomarkers. |
| 4 | The panel of statement 1, wherein the panel comprises 3 |
| | biomarkers. |
| 5 | The panel of statement 1, wherein the panel comprises 4 |
| | biomarkers. |
| 6 | The panel of statement 1, wherein the panel comprises 5 |
| | biomarkers. |
| 7 | The panel of statement 1, wherein the panel comprises 6 |
| | biomarkers |
| 8 | The panel of statement 1, wherein the panel comprises 7 |
| | biomarkers |
| 9 | The panel of statement 1, wherein the panel comprises 8 |
| | biomarkers |
| 10 | The panel of statement 1, wherein the panel comprises 9 |
| | biomarkers |
| 11 | The panel of statement 1, wherein the panel comprises 10 |
| | biomarkers |
| 12 | The panel of statement 1, wherein the panel comprises 11 |
| | biomarkers |
| 13 | The panel of statement 1, wherein the panel comprises 12 |
| | biomarkers |
| 14 | The panel of statement 1, wherein the panel comprises 13 |
| | biomarkers |
| 15 | The panel of any one of statements 1-14, wherein the panel |
| | comprises Hexanal. |
| 16 | The panel of any one of statements 1-15, wherein the panel |
| | comprises Heptanal. |
| 17 | The panel of any one of statements 1-16, wherein the panel |
| | comprises Octanal. |
| 18 | The panel of any one of statements 1-17, wherein the panel |
| | comprises Decanal. |
| 19 | The panel of any one of statements 1-18, wherein the panel |
| | comprises Benzaldehyde. |
| 20 | The panel of any one of statements 1-19, wherein the panel |
| | comprises Phenylacetaldehyde. |
| 21 | The panel of any one of statements 1-20, wherein the panel |
| | comprises Undecane. |
| 22 | The panel of any one of statements 1-21, wherein the panel |
| | comprises Benzoic acid. |
| 23 | The panel of any one of statements 1-22, wherein the panel |
| | comprises acetophenone. |
| 24 | The panel of any one of statements 1-23, wherein the panel |
| | comprises dodecanal. |
| 25 | The panel of any one of statements 1-24, wherein the panel |
| | comprises acetic acid. |
| 27 | The panel of any one of statements 1-25, wherein the panel |
| | comprises phenol. |
| 28 | The panel of any one of statements 1-26, wherein the panel |
| | comprises acetone. |

TABLE 7

| Statement | Biomarkers |
|---|---|
| 1 | A panel of biomarkers selected from the group comprising |
| | Hexanal, Heptanal, Octanal, Decanal, Benzaldehyde, |
| | Phenylacetaldehyde, Undecane and Benzoic acid (i.e. 8 |
| | biomarkers) |
| 2 | The panel of statement 1, wherein the panel comprises 9 |
| | biomarkers |
| 3 | The panel of statement 1, wherein the panel comprises 10 |
| | biomarkers |
| 4 | The panel of statement 1, wherein the panel comprises 11 |
| | biomarkers |
| 5 | The panel of statement 1, wherein the panel comprises 12 |
| | biomarkers |
| 6 | The panel of statement 1, wherein the panel comprises 13 |
| | biomarkers |
| 7 | The panel of any one of statements 1-6, wherein the panel |
| | comprises acetophenone. |
| 8 | The panel of any one of statements 1-7, wherein the panel |
| | comprises dodecanal. |
| 9 | The panel of any one of statements 1-8, wherein the panel |
| | comprises acetic acid. |
| 10 | The panel of any one of statements 1-9, wherein the panel |
| | comprises phenol. |
| 11 | The panel of any one of statements 1-10, wherein the panel |
| | comprises acetone. |

What is claimed is:

1. A method of detecting and treating a cancer in a subject, the method comprising the step of a) detecting or measuring a panel of biomarkers comprising Hexanal, Heptanal, Octanal, Decanal, Benzaldehyde, Phenylacetaldehyde, Undecane and Benzoic acid in a sample obtained from the subject; and b) administering an anti-cancer therapy to the subject found to have cancer or likely to have cancer.

2. The method of claim 1, wherein an increased level of each of the biomarker as compared to a reference indicates the presence of cancer in the subject.

3. The method of claim 1, wherein the method further comprises detecting a biomarker selected from the group consisting of Acetophenone, Dodecanal, Acetic acid, Phenol and Acetone.

4. The method of claim 1, wherein the panel of biomarkers comprises Hexanal, Heptanal, Octanal, Decanal, Dodecanal, Benzaldehyde, Phenylacetaldehyde, Phenol, Acetophenone, Undecane, Acetic acid, Acetone and Benzoic acid.

5. The method of claim 4, wherein the subject is found to have cancer or likely to have cancer if 3 or more of the following conditions are met:
a) Hexanal concentration is at least 1.2 times of the control;
b) Heptanal concentration is at least 1.5 times of the control;
c) Octanal concentration is at least 1.6 times of the control;
d) Decanal concentration is at least 1.7 times of the control;
e) Dodecanal concentration is at least 1.3 times of the control;
f) Benzaldehyde concentration is at least 1.2 times of the control;
g) Phenylacetaldehyde concentration is at least 1.2 times of the control;
h) Phenol concentration is at least 1.1 times of the control;
i) Acetophenone concentration is at least 1.2 times of the control;
j) Undecane concentration is at least 1.3 times of the control;
k) Acetic acid concentration is at least 1.2 times of the control;
l) Acetone concentration is below 0.9 times of the control; or
m) Benzoic Acid concentration is at least 2 times of the control.

6. The method of claim 4, wherein an increased level of Hexanal, Heptanal, Octanal, Decanal, Dodecanal, Benzaldehye, Phenylacetaldehyde, Phenol, Acetophenone, Undecane, Acetic acid, Benzoic acid, and a decreased level of acetone, as compared to a control indicates the presence or likelihood of cancer in the subject.

7. The method of claim 1, wherein the cancer is a lung cancer.

8. The method of claim 1, wherein the sample is exhaled breath.

9. The method of claim 1, wherein the control is a subject or group of subjects without cancer.

10. (canceled)

11. The method of claim 1, wherein the method further comprises detecting a symptom of cancer in the subject.

12. (canceled)

13. A method of detecting and treating of tuberculosis in a subject, the method comprising the step of a) detecting or measuring a panel of biomarkers comprising Hexanal, Heptanal, Octanal, Decanal, Benzaldehyde, Phenylacetaldehyde, Undecane and Benzoic acid in a sample obtained from the subject; and b) administering an anti-tuberculosis therapy to the subject found to have tuberculosis or likely to have tuberculosis.

14. The method of claim 11, wherein the method further comprises detecting a biomarker selected from the group consisting of Acetophenone, Dodecanal, Acetic acid, Phenol and Acetone.

15. The method of claim 11, wherein the panel of biomarkers comprises Hexanal, Heptanal, Octanal, Decanal, Dodecanal, Benzaldehyde, Phenylacetaldehyde, Phenol, Acetophenone, Undecane, Acetic acid, Acetone and Benzoic acid.

16. The method of claim 13, wherein the subject is found to have tuberculosis or likely to have tuberculosis if 3 or more of the following conditions are met:
a) Hexanal concentration is at least 1.1 times of the control;
b) Heptanal concentration is at least 1.1 times of the control;
c) Dodecanal concentration is below 0.7 times of the control;
d) Benzaldehyde concentration is below 0.8 times of the control;
e) Phenylacetaldehyde concentration is below 0.7 times of the control;
f) Phenol concentration is below 0.7 times of the control;
g) Acetophenone concentration is at below 0.8 times of the control;
h) Acetic acid concentration is at below 0.7 times of the control group; or
i) Benzoic Acid concentration is at least 2 times of the control group.

17. The method of claim 11, wherein the control is a subject or group of subjects without tuberculosis.

18. (canceled)

19. A method of differentiating the presence of tuberculosis from cancer in a subject and treating the tuberculosis, the method comprising the steps of a) detecting or measuring a panel of biomarkers comprising Hexanal, Heptanal, Octanal, Decanal, Benzaldehyde, Phenylacetaldehyde, Undecane and Benzoic acid in a sample obtained from the subject; and b) treating the tuberculosis.

20. The method of claim 16, wherein the method further comprises detecting a biomarker selected from the group consisting of Acetophenone, Dodecanal, Acetic acid, Phenol and Acetone.

21. The method of claim 16, wherein the panel of biomarkers comprises Hexanal, Heptanal, Octanal, Decanal, Dodecanal, Benzaldehyde, Phenylacetaldehyde, Phenol, Acetophenone, Undecane, Acetic acid, Acetone and Benzoic acid.

22. The method of claim 16, wherein the control is a subject or a group of subjects without cancer or tuberculosis.

23. The method of claim 16, wherein any one of the following conditions indicates the presence or likelihood of tuberculosis and not cancer:
(a) a comparable level of Octanal to control;
(b) a comparable level of Decanal to control;
(c) a Dodecanal level of less than 0.7 times of the control;
(d) a Benzaldehyde level of less than 0.8 times of the control;
(e) a Phenylacetaldehyde level of less than 0.7 times of the control,
(g) a Phenol concentration of less than 0.7 times of the control;
(h) an Acetophenone concentration of less than 0.8 times of the control; or
(i) an Acetic acid level of less than 0.7 times of the control.

24. (canceled)

25. (canceled)

26. (canceled)

27. (canceled)
